(19) United States
(12) Patent Application Publication
(10) Pub. No.: US 2025/0006332 A1
(43) Pub. Date: Jan. 2, 2025

(54) Probabilistic Graphical Model-Based Prediction of Outcomes in the Treatment of Major Depressive Disorder in Adolescents

(71) Applicant: Mayo Foundation for Medical Education and Research, Rochester, MN (US)

(72) Inventors: Arjun P. Athreya, Rochester, MN (US); William V. Bobo, Jacksonville, FL (US); Liewei Wang, Rochester, MN (US); Richard M. Weinshilboum, Rochester, MN (US); Paul E. Croarkin, Rochester, MN (US)

(21) Appl. No.: 18/690,340

(22) PCT Filed: Sep. 8, 2022
(86) PCT No.: PCT/US2022/042968

§ 371 Date: Mar. 8, 2024

Related U.S. Application Data
(60) Provisional application No. 63/241,704, filed on Sep. 8, 2021.

(51) Int. Cl. G16H 20/10; G16H 10/20; G16H 10/60

(52) U.S. Cl. CPC G16H 20/10; G16H 10/20; G16H 10/60

(57) ABSTRACT

Likely outcomes of a treatment of an adolescent patient who has major depressive disorder (“MDD”) using machine learning. Symptoms reported at one or two points in time are input to a suitably trained machine learning model, generating output that indicate a prediction of the most likely outcome of a particular treatment at a third point in time, a future severity of symptoms, or another clinical course of action. The treatment outcome can include the likelihood of remission, response to drug, selection of the drug most likely to give positive outcomes, and so on. The machine learning model can implement a probabilistic graphical model, as an example.

### STATEMENT REGARDING FEDERALLY SPONSORED RESEARCH

This invention was made with government support under MH113700 and MH124655 awarded by the National Institutes of Health, and under 2041339 awarded by the National Science Foundation. The government has certain rights in the invention.

### BACKGROUND

The treatment of major depressive disorder (“MDD”) in adolescents is an important public health challenge with ongoing controversies regarding the efficacy and safety of antidepressants in pediatric populations, including children and adolescents. Fluoxetine and escitalopram are currently the only FDA approved medications for MDD in adolescents, but other selective serotonin reuptake inhibitors (“SSRIs”) and serotonin norepinephrine reuptake inhibitors (“SNRIs”) are commonly prescribed off-label in clinical practice. Response to antidepressant therapy in adolescents is more heterogeneous when compared to adults. Major treatment planning challenges for adolescents with MDD include determining which adolescents will benefit from acute antidepressant treatment, dosage increases, continuation treatment, and maintenance treatment. There is also a pressing need to improve clinical practice algorithms in primary care environments for the treatment of MDD in adolescents.

One prior study of adolescents in treatment for MDD, described by R. Tao, et al., in “Early Prediction of Acute Antidepressant Treatment Response and Remission in Pediatric Major Depressive Disorder,” J Am Acad Child Adolesc Psychiatry, 2009; 48:71-78, used logistic regression models to demonstrate that the rate of overall depressive symptom improvement was prognostic for acute treatment response. This finding, albeit important, did not capture the inherent heterogeneity of symptom presentations and treatment trajectories.

Recent work described by J. Bondar, et al., in “Symptom Clusters in Adolescent Depression and Differential Response to Treatment: A Secondary Analysis of the Treatment for Adolescents with Depression Study Randomised Trial,” Lancet Psychiatry, 2020); 7:337-343, examined symptoms clusters in adolescents treated for MDD and identified two unique symptoms profiles. One profile demonstrated differential changes in depressive symptoms. A second profile failed to demonstrate differences among active and placebo treatments. These two prior studies did not provide thresholds of improvement in specific symptoms to derive interpretable predictions of acute treatment course of MDD in adolescents, nor did these studies achieve cross-trial replications.

Interpretable predictions for adolescents in treatment for MDD based on symptom changes would catalyze the development of decision support tools that could then be operationalized in specialty or primary care settings.

### SUMMARY OF THE DISCLOSURE

The present disclosure addresses the aforementioned drawbacks by providing a computer-implemented method for predicting a treatment outcome response for an adolescent patient to a particular treatment for major depressive disorder. The method includes accessing symptom measure data for the adolescent patient using the computer system. The symptom measure data include: a first symptom measure for the adolescent patient corresponding to a severity of major depressive disorder at a first point in time; and a second symptom measure for the adolescent patient corresponding to a severity of major depressive disorder at a second point in time that is subsequent to the first point in time. A first symptom class and a second symptom class are selected with the computer system based on the symptom measure data. The first symptom class is selected from a first set of symptom classes corresponding to a first symptom range that contains the first symptom measure, and the second symptom class is selected from a second set of symptom classes corresponding to a second symptom range that contains the second symptom measure. The first symptom class and the second symptom class are input to a trained machine learning model using the computer system, generating output as a prediction of a treatment outcome for the adolescent patient in response to a particular treatment regimen. An indication of the prediction of the treatment outcome can then be provided using the computer system.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1A illustrates an example implementation of a probabilistic graphical model (“PGM”)-based tool for deriving prognoses of treatment outcomes in adolescents treated with fluoxetine or duloxetine.

FIG. 1B illustrates example trajectories of Children's Depression Rating Scale-Revised (“CDRS-R”) total scores in patients treated with fluoxetine.

FIG. 2A illustrates an example construction of a probabilistic graphical model (“PGM”) using a hidden Markov model that includes hidden states, observation states, and transitions. The hidden states are defined using ranges of total depression severity, where the ranges for depression severity at baseline are inferred using unsupervised machine learning. Observation states at the treatment's intermediate (4 to 6 weeks) or endpoint (10 to 12 weeks) record active depression (i.e., CDRS-R total depression severity score >40).

FIG. 2B is an example of a compact representation of CDRS-R total score variations derived using PGM. In this example, patients starting in any stratum at baseline were most likely to achieve remission (i.e., be in C1 strata) at 10 to 12 weeks if they transitioned into the B1 stratum at 4 to 6 weeks, and the clinical observation at 4 to 6 weeks was also remission. Patients starting in the A2 strata at baseline were most likely to achieve response at 10 to 12 weeks if they transitioned into the B2 or B3 stratum at 4 to 6 weeks and the clinical observation at 4 to 6 weeks was response or non-response respectively; and were most likely to be non-responders at 10 to 12 weeks if they transitioned into the B3 stratum at 4 to 6 weeks and the clinical observation at 4 to 6 weeks was also a non-response. Patients starting in the A1 stratum at baseline were most likely to be non-responders at 10 to 12 weeks if they transitioned into the B2 stratum at 4 to 6 weeks and the clinical observation at 4 to 6 weeks was also non-response.

FIG. 2C shows an example symptom cluster derived using hierarchical clustering in A2 stratum of patients with CDRS-R depression severity >56.

FIG. 3 illustrates an example machine learning workflow that can be implemented in accordance with some embodiments described in the present disclosure.

FIG. 4 is a flowchart setting forth the steps of an example method for predicting a treatment outcome for an adolescent patient having major depressive disorder to a particular treatment regimen using a PGM-based machine learning model.

FIGS. 5A-5D shows the variation in the severity of irritability (CDRS-R item 4) in patients originating from A1 stratum at baseline in an example study. Though the band of the shaded region is narrow around the mean indicating reduction in symptom severity in response to therapy, the height of the boxplot and the extent of whiskers indicate high-degrees of variability in scores at each timepoint.

FIG. 5B shows the variation in prognostic symptoms (e.g., irritability) and non-prognostic symptoms (e.g., impaired school work) in symptom dynamic paths of patients originating in A1 stratum and treated with fluoxetine in an example study

FIG. 5C shows variations of non-prognostic symptoms (e.g., impaired school work) in symptom dynamic paths of patients originating in A1 stratum and treated with a placebo in an example study.

FIG. 5D shows the variation in prognostic symptoms (e.g., irritability) and non-prognostic symptoms (e.g., impaired school work) in symptom dynamic paths of patients originating in A1 stratum and treated with duloxetine in an example study.

FIG. 5E shows the variation of prognostic and non-prognostic symptom severity of CDRS-R scale on symptom dynamic paths originating from A1 stratum at baseline (i.e., A1 B1 C1, A1 B2 C2 and A1 B3 C3) in patients treated with fluoxetine in an example study.

FIG. 6 is a block diagram of an example system for predicting a treatment outcome for an adolescent patient using the methods described in the present disclosure.

FIG. 7 is a block diagram of example components that can implement the system of FIG. 6.

### DETAILED DESCRIPTION

Described here are systems and methods for predicting likely outcomes of a treatment of a pediatric patient who has major depressive disorder (“MDD”) using machine learning. The pediatric patient may be an adolescent, as an example. By inputting symptoms reported at one or two points in time to a suitably trained machine learning algorithm or model, the systems and methods described in the present disclosure can generate output indicating a prediction of the most likely outcome of the treatment at a third point in time. Additionally or alternatively, the generated output can include a score, feature vector, or report indicating the likelihood of the most likely treatment outcome at the third point in time. The treatment outcome can include the likelihood of remission, response to drug, selection of the drug most likely to give positive outcomes, and so on. In some implementations, additional data can be input to the trained machine learning algorithm or model, including information from metabolomic data and/or genomic data.

The systems and methods described in the present disclosure make use of probabilistic graphical models (“PGMs”) coupled with unsupervised machine learning techniques focused on depressive symptoms to develop accurate and valid prognostic information in adolescents undergoing treatment for MDD. Advantageously, the use of PGMs allows for the degrees of accuracies in predictions of treatment outcomes achieved in depressed adults despite the increased inter-patient variability observed in treatment outcomes in depressed adolescents.

Advantageously, the systems and methods described in the present disclosure utilize a rating scale to assess symptoms, particular outcomes studied in the adolescent age group, symptom dynamics particular to the adolescent age group together with a unique implementation of machine learning in order to generate the predictive scores and/or reports mentioned above. Additionally, the systems and methods can implement an evaluation of patient response to placebo.

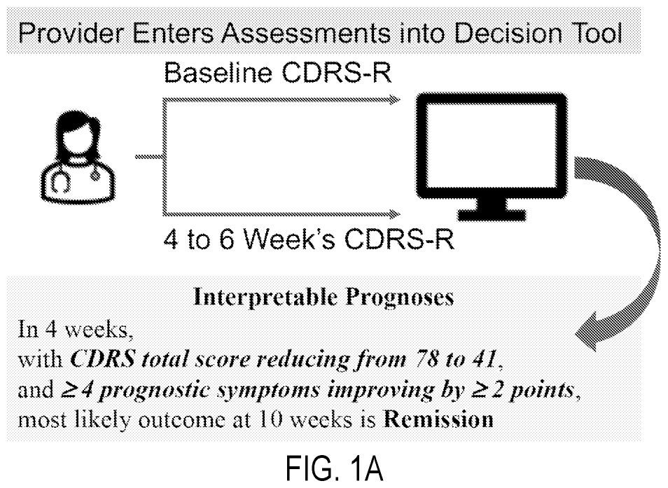

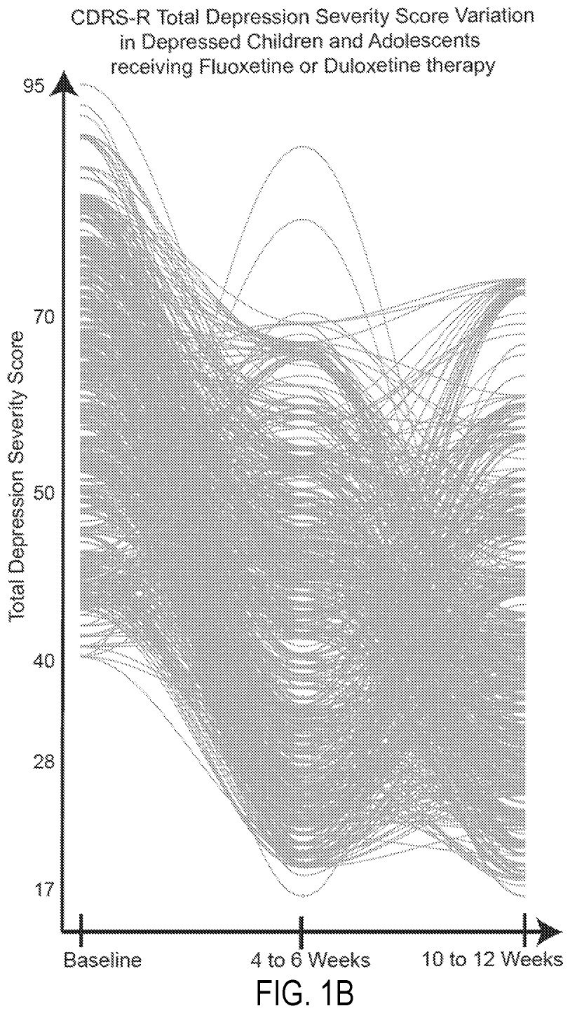

An example implementation of a PGM-based tool for deriving prognoses of treatment outcomes in adolescents treated with fluoxetine or duloxetine is shown in FIG. 1A. In this example, the user inputs patient data for a first time point (e.g., baseline) and optionally patient data for a second time point (e.g., 4-6 weeks) and the systems and methods described in the present disclosure generate output as a report indicating a likelihood of treatment outcome at a later time point (e.g., 10-12 weeks).

In general, a PGM provides a framework for encoding multivariate probability distributions using one or more graphs that are capable of capturing conditional relationships between interacting random variables. By knowing the graph structure of a PGM, tasks such as inference and/or learning can be completed. As such, the PGM can be used to compute marginal distributions of random variables and/or to estimate parameters of probability functions.

As a non-limiting example, a graphical model can be represented by a graph with its nodes, which may be also be referred to as vertices, corresponding to random variables and its edges corresponding to dependency relationships between the nodes. A PGM can be constructed to include nodes corresponding to states that represent MDD severity at each time point of treatment and a probability of transitions between states (e.g., fraction of patients moving between states of consecutive time points, such as baseline to 4-6 weeks). For each time point, t, if it is assumed that there are N unique CDRS-R scores, then the number of trajectories of scores is proportional to N^{t}. FIG. 1B illustrates example trajectories of CDRS-R total scores in adolescent patients treated with fluoxetine.

In an example configuration, patients can be stratified or otherwise grouped in order to reduce N, and to derive a compact representation of antidepressant response in adolescents. Three strata, or groups, can be defined at treatment intermediate or endpoints based on symptom severity thresholds: a first group for patients in active depression, a second group for patients in remission, or a third group for patients with CDRS-R scores greater than 28 and less than 40.

For baseline stratification, an unsupervised learning approach can be used to derive patient strata, or groups. As an example, the distribution of CDRS-R scores is likely to be characterized by multiple Gaussian curves. In these instances, Gaussian mixture models can be chosen as the unsupervised learning approach.

In an example implementation, CDRS-R total scores from a baseline group treated only with fluoxetine can be input to the unsupervised learning algorithm described above, generating output as two groups of patients (patient clusters) inferred in the training datasets based on total CDRS-R scores at baseline. In this example, the letters A, B, and C represent the treatment timepoints and the numeric suffix at each timepoint represents the level of depression severity, with “3” being the most severely depressed patients and “1” being the least-severely depressed. The ranges of total CDRS-R scores for each cluster in this example were as follows: baseline group: A1 [≤55], A2 [≥56]; intermediate timepoint (e.g., 4-6 weeks) group: B1 [0-28], B2 [29-39], B3 [≥40]; and endpoint (e.g., 10-12 weeks) group: C1 [0-28], C2 [29-39], and C3 [≥40].

In comparison with 397 unique trajectories shown in the example of FIG. 1B, by stratifying patients at each time point it is contemplated that a maximum number of MDD response trajectories can be significantly reduced, such as to 18 paths in this example (i.e., N=2 at baseline, N=3 at both the intermediate timepoint and endpoint, such that N^{t}=2*3²=18).

In an example implementation, the PGM can be constructed using a hidden Markov model (“HMM”) formulation to extract the most likely variation in depression severity during the treatment for patients starting from a given baseline strata. For each treatment time point, the HMM can be characterized by the following: hidden states (patient strata defined by range of total CDRS-R score); observation states at the intermediate timepoint and endpoint (response status of patients in respective strata); and forward transition probabilities (fraction of patients moving between strata of one timepoint to the next timepoint).

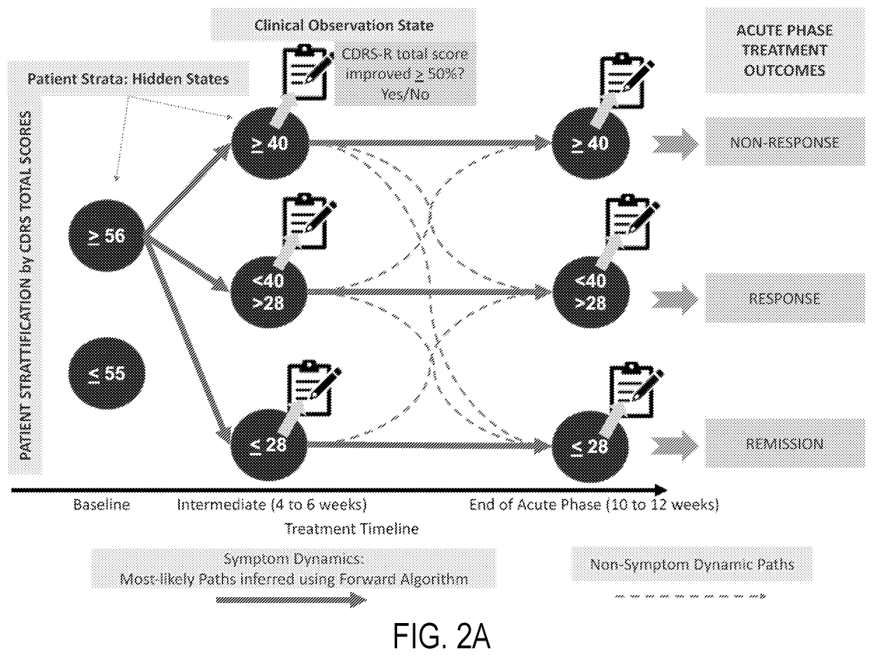

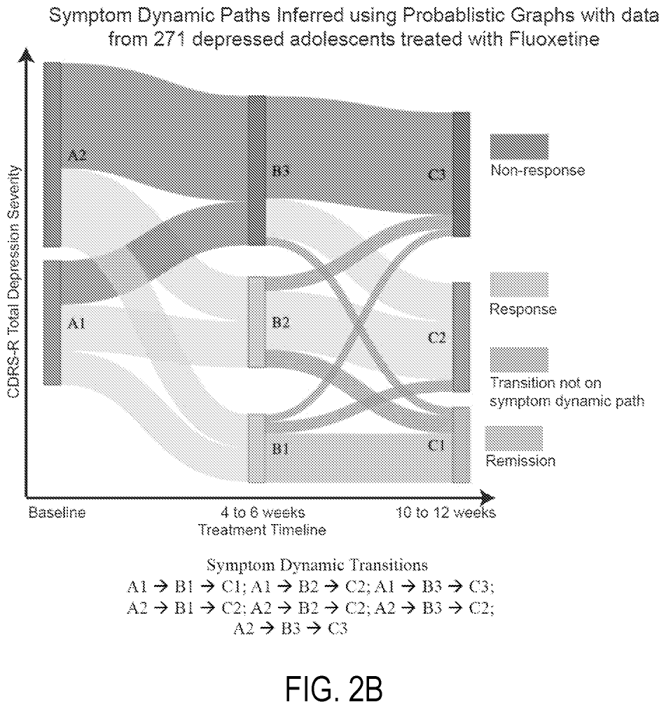

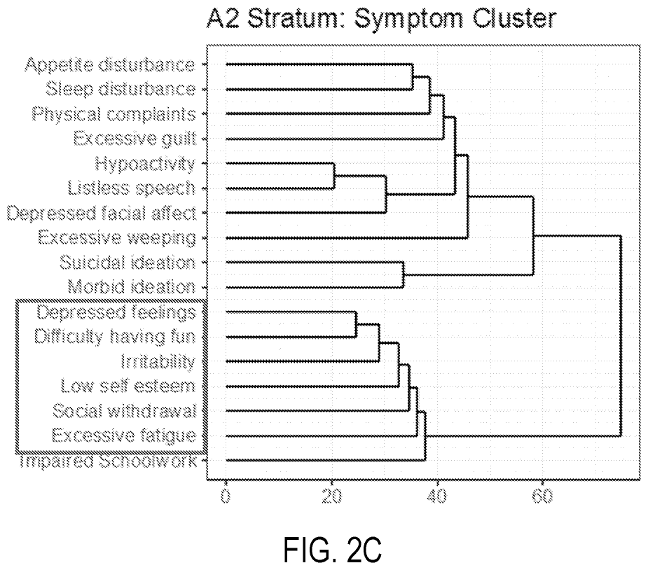

FIG. 2A shows an example PGM using an HMM that includes hidden states, observation states, and transitions. As described above, the hidden states can be defined using ranges of total depression severity, where the ranges for depression severity at baseline can be inferred using unsupervised machine learning. Observation states at the treatment's intermediate (e.g., 4 to 6 weeks) or endpoint (10 to 12 weeks) record active depression (i.e., CDRS-R total depression severity score >40).

The forward algorithm helps derive likelihood for all paths originating from a baseline stratum and ending in an endpoint group (e.g., a 10-12 weeks group) without having to condition the trajectories on a specific outcome of interest (e.g., remission). For every pair of groups at baseline and an endpoint (e.g., 10-12 weeks), the paths that have the highest likelihood and a threshold amount of the patients from the baseline group can be selected as the symptom dynamic paths. As a non-limiting example, the threshold amount of patients can be at least 10 percent of the patients from the baseline group.

As a non-limiting example, a forward algorithm can be used to identify the most likely forward transitions a patient starting in any baseline group will make between groups (hidden states C) of the trial, and also what the associated clinical outcomes are likely to be during the transitions (observed states O). During transitions between the groups, the clinician/psychiatrist assessing the patient observes the clinical outcome O={O_{R}, O_{NR}}, which is whether the patient has demonstrated either response or no-response. The symptom dynamics for any patient, starting in any of the groups at baseline, can be predicted recursively using the forward algorithm. The forward algorithm can be described as:

$${P}_{O} ( {C}_{t} ) = {\sum }_{t ∈ T} p ( O \vert {C}_{t} ) {P}_{O} ( {C}_{t - 1} ) p ( {C}_{t - 1} \to {C}_{t} )$$

where p(O|Cₜ) is the probability of the observation (response or no-response) in a current state, p(Cₜ₋₁→Cₜ) is the probability of a transition from a state of a previous timepoint to a state of the current timepoint, and P_{O}(Cₜ₋₁) is the path probability for a given set of observations seen until Cₜ₋₁. Implementing an HMM can allow for the symptom dynamics to be modeled not only as a function of how symptoms themselves change, but also as a function of potentially associated clinical outcomes during various timepoints of the trial or treatment regimen.

In Table 1 below, for each path the path likelihood used to identify symptom dynamic paths is illustrated. Symptom dynamic paths between a baseline and 10 to 12 week (e.g., endpoint) strata are highlighted in green based on highest likelihood score. For example, the symptom dynamic path between A1 and C1, is A1→B1→C1 as the path has likelihood greater than other paths between A1 and C1. The ranges of depression severity scores in each stratum in this example were as follows: A1 [<55], A2 [>56]; B1 [<28], B2 [>29 and <39], B3 [>40] and C1 [<28], C2 [>29 and <39], C3 [>40].

FIG. 2B shows an example of a compact representation of CDRS-R total score variations derived using a PGM. In this example, patients starting in any stratum at baseline were most likely to achieve remission (i.e., to be in the C1 strata) at 10 to 12 weeks if they transitioned into the B1 stratum at 4 to 6 weeks, and the clinical observation at 4 to 6 weeks was also remission. Patients starting in the A2 strata at baseline were most likely to achieve response at 10 to 12 weeks if they transitioned into the B2 or B3 stratum at 4 to 6 weeks and the clinical observation at 4 to 6 weeks was response or non-response respectively; and were most likely to be non-responders at 10 to 12 weeks if they transitioned into the B3 stratum at 4 to 6 weeks and the clinical observation at 4 to 6 weeks was also a non-response. Patients starting in the A1 stratum at baseline were most likely to be non-responders at 10 to 12 weeks if they transitioned into the B2 stratum at 4 to 6 weeks and the clinical observation at 4 to 6 weeks was also non-response.

Prognostic symptoms can be defined to meet at least the following criteria. A first example criterion includes non-zero symptom severities at baseline across the majority of patients (to assess the quantum of early reductions in severity during treatment for predicting long-term response). A second example criterion includes similar symptom severity scores. Symptom clusters can be created by deriving the clusters using hierarchical clustering for each stratum at all time points on symptom dynamic paths originating from a baseline stratum. An example symptom cluster for an A2 stratum is shown in FIG. 2C. Creating these symptom clusters can establish how many symptoms with similar severity at baseline should improve at 4 to 6 weeks for predicting 10 to 12-week outcomes. A third example criterion can include different distributions of symptom severity scores between symptom dynamic paths (e.g., to quantify the level of change in a group of symptoms at 4 weeks needed to achieve specific outcomes at 10 to 12 weeks).

These criteria help identify a set of depressive symptoms that have similar severities at baseline (e.g., the first two criteria described above) and across individual time points (e.g., a grouping effect), and have different levels of severity between individual symptoms dynamic pathways (e.g., a discriminatory effect, with the third criterion described above).

For prognostic symptoms, prognoses rules can be defined. For example, prognoses rules can include a threshold of change at an intermediate timepoint (e.g., 4 to 6 weeks) and how many symptoms should exceed (or not) to achieve an outcome at an endpoint (e.g., 10 to 12 weeks). Using absolute difference in median scores of prognostic symptoms on symptom dynamic paths from baseline to intermediate strata can be used to derive thresholds of change needed to achieve a certain outcome of interest. Examples of median symptom severity scores on symptom dynamic paths are shown in Table 2.

TABLE 2

| Symptom Dynamic Path | A1 −> B1 −> C1 | A1 −> B2 −> C2 | A1 −> B3 −> C3 | A2 −> B1 −> C1 |
|---|---|---|---|---|
| Difficulty having fun | 4 −> 1 −> 1 | 4 −> 2 −> 2 | 5 −> 5 −> 4 | 6 −> 1 −> 1 |
| Social withdrawal | 3 −> 1 −> 1 | 4 −> 2 −> 1 | 3 −> 5 −> 3 | 5 −> 1 −> 1 |
| Exessive fatigue | 5 −> 1 −> 1 | 5 −> 2 −> 3 | 5 −> 6 −> 4 | 5 −> 1 −> 1 |
| Irriability | 5 −> 1 −> 1 | 4 −> 2 −> 2 | 5 −> 3 −> 5 | 5 −> 1 −> 1 |
| Low self esteem | 3 −> 1 −> 1 | 4 −> 3 −> 3 | 3 −> 5 −> 5 | 5 −> 1 −> 1 |
| Depressed Feelings | 5 −> 1 −> 1 | 4 −> 3 −> 2 | 4 −> 5 −> 3 | 6 −> 1 −> 1 |

| Symptom Dynamic Path | A2 −> B2 −> C2 | A2 −> B3 −> C2 | A2 −> B3 −> C3 |
|---|---|---|---|
| Difficulty having fun | 6 −> 2 −> 1 | 5 −> 3 −> 3 | 5 −> 3 −> 3 |
| Social withdrawal | 5 −> 2 −> 1 | 5 −> 3 −> 3 | 5 −> 3 −> 3 |
| Exessive fatigue | 6 −> 3 −> 2 | 5 −> 5 −> 3 | 5 −> 5 −> 3 |
| Irriability | 5 −> 3 −> 2 | 5 −> 3 −> 3 | 5 −> 5 −> 4 |
| Low self esteem | 4 −> 3 −> 2 | 5 −> 4 −> 2 | 5 −> 3 −> 3 |
| Depressed Feelings | 5 −> 2 −> 1 | 5 −> 3 −> 2 | 5 −> 5 −> 3 |

Chi-square tests can be used to identify the minimum number of prognostic symptoms needed to (or not) exceed thresholds at an intermediate timepoint to be prognostic of outcomes at an endpoint.

For the prognoses rules associated with each baseline and intermediate transition, accuracy and odds ratio (“OR”) of the most-likely outcome expected at endpoint can be reported. An example of prognoses performance of prognostic symptoms in patients treated with fluoxetine making specific transitions between baseline and 4 to 6 week strata is shown in Table 2. The ranges of depression severity scores in each stratum are as follows: A1 [<55], A2 [>56]; B1 [<28], B2 [>29 and <39], B3 [>40]. The OR represents the odds that the expected treatment outcome at 8 weeks will occur if patients are covered by the prognoses rule, compared to the odds of the same outcome occurring in patients not covered by the prognoses rule. The statistical significance (p-value) of the prognoses' accuracy was established using the null information rate (“NIR”). Table 3 shows an example prognoses performance in patients treated with duloxetine and placebo.

TABLE 3
TRAINING (TADS and Eli Lilly) Fluoxetine N = 271

| | | | | Prognoses Rule and Coverage | | |
|---|---|---|---|---|---|---|
| | | | | | | Coverage |
| | | | | Change in | | (Fraction of |
| | Intermediate | Number of | | symptom | Number of | patients |
| | timepoint | patients | Most likely | severity | symptoms | covered by |
| Baseline | strata (4 to 6 | making | outcome at 10 | (Baseline - 4 | needing the | prognoses |
| strata | weeks) | transition | to 12 weeks | to 6 weeks) | change | rule) |
| A2 | B3 | 83 | Non-response | <=2 | >=4 | 0.80 |
| | B2 | 38 | Response | >=2 | >=3 | 0.92 |
| | B1 | 35 | Remission | >=2 | >=4 | 0.97 |
| A1 | B3 | 46 | Non-response | <=1 | >=5 | 0.71 |
| | B2 | 42 | Response | >=1 | >=4 | 0.73 |
| | B1 | 27 | Remission | >=2 | >=4 | 0.77 |

| | Probablity of most | p-value of | | |
|---|---|---|---|---|
| | likely outcome | accuracy | | 95% |
| Baseline | (Accuracy = | with NIR = | Odds Ratio | Confidence |
| strata | 100*Probabilty) | 0.52 | (OR) | interval |
| A2 | 0.62 | 0.06 | 12.30 | (25, 58.3) |
| | 0.86 | 6.80E−05 | 8.40 | (1.1, 63.7) |
| | 0.67 | 0.09 | 8.00 | (0.78, 81) |
| A1 | 0.66 | 0.007 | 7.00 | (1.6, 30.4) |
| | 0.84 | 0.005 | 1.15 | (0.2, 7) |
| | 0.72 | 0.05 | 8.30 | (12, 55.3) |

TABLE 4
VALIDATION STUDY

| | | | | Prognoses Rule and Covenge | | | | |
|---|---|---|---|---|---|---|---|---|
| | | | | | | Coverage | | |
| | | | | Change in | | (Fraction of | | |
| | Intermediate | Number of | | symptom | Number of | patients | Probablity of most- | p-value of |
| | timepoint | patients | Most likely | severity | symptoms | covered by | likely outcome | accuracy |
| Baseline | strata (4 to 6 | making | outcome at 10 | (Baseline - 4 | needing the | prognoses | (Accuracy = | with NIR = |
| strata | weeks) | bansition | to 12 weeks | to 6 weeks) | change | rule) | 100*Probabilty) | 0.52 |

TESTING: Prognoses Performance at 4 weeks in Duloxetine treated Patients (Eli Lilly N = 255)

| | | | | | | | | |
|---|---|---|---|---|---|---|---|---|
| A2 | B3 | 97 | Non-response | <=2 | >=4 | 0.85 | 0.63 | 0.02 |
| | B2 | 38 | Response | >=2 | >=3 | 0.86 | 0.84 | 6.61E−05 |
| | B1 | 23 | Remission | <=2 | >=4 | 0.78 | 0.83 | 0.007 |
| A1 | B3 | 30 | Non-response | <=1 | >=5 | 0.80 | 0.7 | 0.06 |
| | B2 | 39 | Response | >=1 | >=4 | 0.84 | 0.84 | 6.60E−05 |
| | B1 | 30 | Remission | >=2 | >=4 | 0.77 | 0.70 | 0.09 |

TESTING: Prognoses Performance at 4 to 6 weeks in Placebo treated Patients (Eli Lilly and TADS N = 265)

| | | | | | | | | |
|---|---|---|---|---|---|---|---|---|
| A2 | B3 | 119 | Non-response | <=2 | >=4 | 0.90 | 0.58 | 0.22 |
| | B2 | 33 | Response | >=2 | >=3 | 0.93 | 0.77 | 0.003 |
| | B1 | 18 | Remission | >=2 | >=4 | 0.88 | 0.75 | 0.08 |
| A1 | B3 | 39 | Non-response | <=1 | >=5 | 0.62 | 0.38 | 0.3 |
| | B2 | 33 | Response | >=1 | >=4 | 0.85 | 0.78 | 0.003 |
| | B1 | 23 | Remission | >=2 | >=4 | 0.70 | 0.81 | 0.02 |

In Tables 2 and 3, accuracy is the fraction of patients for whom the prognoses rules predicted the correct treatment outcome. The OR represents the odds that the expected treatment outcome at 10 to 12 weeks would occur if patients met the prognoses rule criteria, compared to the odds of the same outcome occurring in patients not meeting the prognoses rule.

A statistical significance (p-value) of accuracies derived using prognoses rules was computed by comparing the accuracy against the NIR, serving as a proxy for chance. An NIR of 0.52 represents the fraction of patients in the training datasets for whom the categorical non-responder status at 4 to 6 weeks correctly predicted active depression at 10 to 12-weeks (i.e., only 52% of the 271 patients in the training data who were non-responders at 4 to 6 weeks [≥40 in total CDRS-R at 4 to 6 weeks] were responders at 10 to 12 weeks; this is, their CDRS-R total score was ≤39). The Kolmogorov-Smirnov (for age) and Chi-square tests (for sex and race) can be used to evaluate if prognosis rules or accuracies were associated with age, sex, or race (the common sociodemographic factors across all datasets).

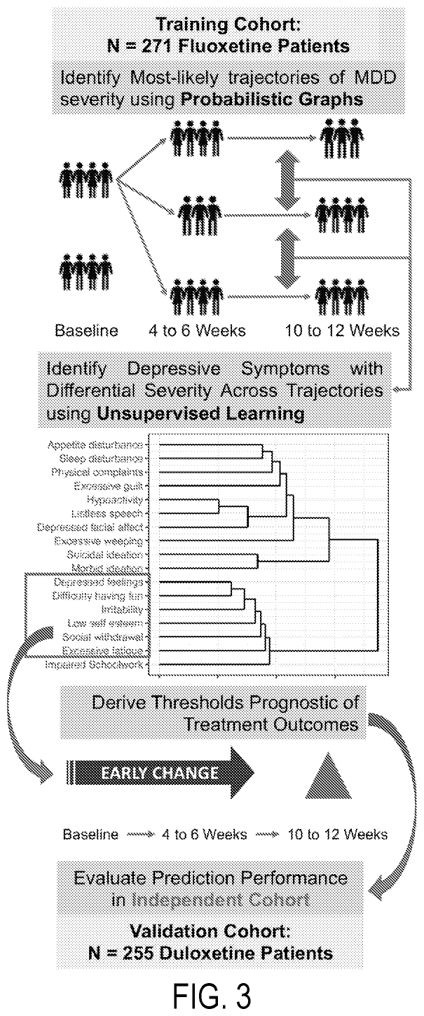

An example workflow for implementing a machine learning algorithm and/or model that implements PGMs to predict a treatment outcome in adolescent patients is shown in FIG. 3. The workflow generally includes selecting training data an identifying the most likely trajectories of MDD severity using PGMs, as described above. Depressive symptoms with differential severity across trajectories can then be identified using unsupervised learning, as described above. Thresholds that are prognostic of treatment outcomes are then derived and can be used to evaluate the prediction performance in an independent cohort.

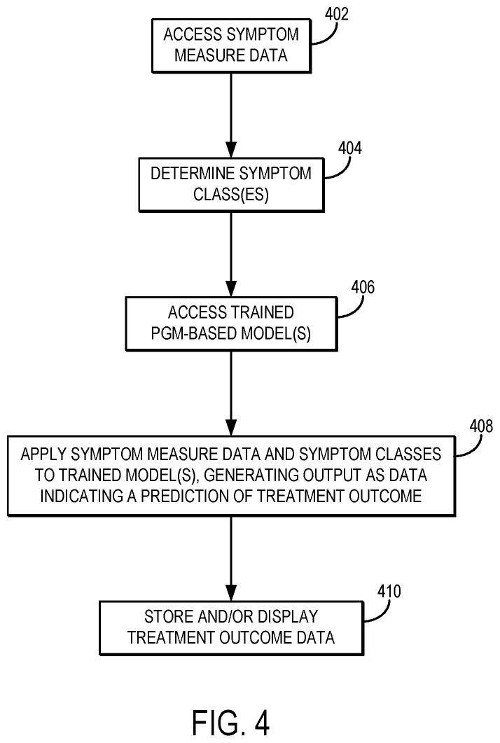

Referring now to FIG. 4, a flowchart is illustrated as setting forth the steps of an example method for predicting a treatment outcome for an adolescent patient having MDD using a suitably trained machine learning algorithm and/or model.

The method includes accessing symptom measure data for an adolescent patient with a computer system, as indicated at step 402. Accessing the symptom measure data may include retrieving such data from a memory or other suitable data storage device or medium. Alternatively, accessing the baseline may include acquiring such data and transferring, entering, or otherwise communicating the data to the computer system.

In general, the symptom measure data can include data representative of patient symptoms that are measured or otherwise recorded at a baseline timepoint (e.g., before beginning a treatment regimen). The symptom measure data may be augmented with other data, including demographic information (e.g., age, level of physical activity, race, income, family history of depression or other diseases or disorders), functional connectivity data, metabolomic data, genomic data, other biological measure data (e.g., information about one or more physiological properties, a body mass index, a blood pressure), or the like.

The symptom measure data can include one or more symptom measures for the adolescent patient, where each symptom measure can correspond to a severity of a symptom of a disease or disorder of the person (e.g., MDD) at respective different points in time (e.g., a pre-treatment baseline point in time, an intermediate point in time 4-6 weeks post-treatment). As described above, the symptom measures can be used to predict a treatment outcome, or response, at a later point in time (e.g., at 10-12 weeks post-treatment) and/or to determine some other clinical course of action (e.g., to adjust a dosage of an anti-depressant or other drug, to change a drug received by the adolescent patient, to select a drug from an enumerated list of potential drugs, to provide a surgery or other therapy).

The symptom measures can be any measure of the severity of one or more symptoms of a disease or disorder of a person. For example, the symptom measure could be a measure of the severity of MDD measured by applying a standard clinical assessment of MDD. As an example, symptom measure data may include a CDRS-R total score value. As another example, the symptom measure data can include Patient Health Questionnaire modified for teens (PHQ-9M) response data. The symptom measure could be a subjective measure (e.g., based on a patient's self-reporting) and/or an objective measure (e.g., a galvanic skin response or other measure of physiological stress, a diastolic and/or systolic blood pressure).

Genomic data can include a nucleotide sequence of the adolescent patient, the presence or absence of a single-nucleotide polymorphism at a particular location within the genome of the adolescent patient, or some other information about the genome or other genetic property (e.g., epigenetic markers) of the adolescent patient.

Metabolomic data can include levels of specified substances in the blood, lymph, saliva, tears, or other fluids of the adolescent patient. Substances of interest (e.g., in predicting response, with respect to the symptoms of major depressive disorder, to treatment with antidepressant drugs) may include (+)-alpha-tocopherol, uric acid, kynurenine, 3-hydroxy kynurenine, alpha-methyltryptophan, indole-3-propionic acid, (+)-gamma-tocopherol, serotonin, methoxy-hydroxyphenyl glycol, methionine, homogentisic acid, or 1,7-dimethylxanthine.

Each symptom measure is then used to determine a corresponding symptom class for a set of symptom classes, as indicated at step 404. Each time period for which a symptom measure is obtained (e.g., pre-treatment, four weeks post-treatment, six weeks post-treatment) has a corresponding set of symptom classes, and each symptom class corresponds to a respective range of observed symptom measure values. The symptom classes for a particular point in time may correspond to clinical populations. For example, the set of symptom classes corresponding to a pre-treatment point in time may correspond to patients exhibiting high, medium, and low levels of pre-treatment symptom severity. In another example, the set of symptom classes corresponding to a post-treatment point in time (e.g., a point in time six weeks post-treatment) may correspond to patients that exhibit no response, response, and remission.

Thus, determining a symptom class for an observed symptom measure includes, based on the observed symptom value, selecting a symptom class from a set of symptom classes for the point in time the corresponds to the observed symptom measure. In some examples, this could include comparing the observed symptom measure to a set of symptom ranges, where each symptom range corresponds to a respective symptom class. The symptom range that contains the observed symptom measure could be determined and the corresponding symptom class selected for the observed symptom measure.

The set of symptom classes and corresponding symptom ranges for a particular point in time could be determined in a variety of ways. For example, an unsupervised learning algorithm can be trained and implemented to determine the set of symptom classes and corresponding symptom ranges. As a non-limiting example, the unsupervised learning algorithm can implement a Gaussian mixture model, as described above, to identify sub-populations within the observed symptom measures for a given point in time.

A trained machine learning model is then accessed with the computer system, as indicated at step 406. Accessing the trained machine learning model may include accessing model parameters (e.g., weights, biases, or both) that have been optimized or otherwise estimated by training the model on training data. In some instances, retrieving the model can also include retrieving, constructing, or otherwise accessing the particular model architecture to be implemented. For instance, data pertaining to the layers in the model architecture (e.g., number of layers, type of layers, ordering of layers, connections between layers, hyperparameters for layers) may be retrieved, selected, constructed, or otherwise accessed. Additionally or alternatively, data pertaining to the symptom dynamics and/or non-symptom dynamic paths can be retrieved, selected, constructed, or otherwise accessed.

In general, the model is trained, or has been trained, on training data in order to estimate or otherwise predict the likelihood of a particular treatment outcome for the patient based on one or more different potential treatment regimens.

The symptom measure data and determined symptom classes are then input to the trained model, generating output as treatment outcome data, as indicated at step 408. The generated treatment outcome data can include a prediction of a particular treatment outcome, a prediction of future symptom severity, and/or some other clinical course of action.

As a non-limiting example, the treatment outcome data may include likelihood scores, classifications of treatment outcome and/or future symptom severity, reports, or other such output. As one example, the treatment outcome data can include a probability that the patient will have a non-response to the treatment, a response to the treatment without remission, or may remission as a result of the treatment.

In this way, using the determined symptom classes and for each observed symptom measure at each point in time for the adolescent patient, a prediction of treatment outcome for the adolescent patient is determined or otherwise estimated. For instance, the treatment outcome can indicate whether the adolescent patient is likely to exhibit remission (e.g., remission of MDD at a time point between ten and twelve weeks post-treatment) in response to a particular treatment (e.g., treatment with fluoxetine, duloxetine, or some other antidepressant drug). Additionally or alternatively, the treatment outcome can indicate the selection a drug from an enumerated list of drugs that is most likely, of the drugs on the list, to result in remission and/or to minimize side effects, or to determine some other clinical course of action. This determination can include predicting a likely symptom measure value or symptom class at a particular future point in time (e.g., predicting whether the adolescent patient person is likely to exhibit no response, to exhibit response, or to exhibit full remission in response to a treatment at a future point in time, such as between ten and twelve weeks post-treatment).

Determining a clinical course of action could additionally or alternatively include generating a suggested change in a dosage of a drug (e.g., an antidepressant drug being provided to the adolescent patient), generating a suggested initial dose of a drug, and/or generating a suggestion that a first drug be discontinued in favor of a different drug. In another example, determining a clinical course of action could include determining that a particular surgical or other therapeutic intervention should be applied. In still another example, determining a clinical course of action could include predicting whether or not the adolescent patient will respond to receiving a placebo treatment.

As described above, a machine learning model that implements, or is based on, a probabilistic graph model can be implemented. As described, such a model can be trained by a set of data that includes, for each one of a plurality of adolescent patients, observed symptom severity measures at a number of different points in time relative to a particular treatment (e.g., the prescription of an antidepressant) as well as the eventual outcome of that treatment (e.g., a symptom severity measure at a particular point in time post-treatment, a changed dose or identity of a treatment drug). Such data could also be used to determine symptom classes and corresponding symptom ranges for each of the observed points in time.

The treatment outcome data generated by inputting the baseline patient to the trained model(s) can then be displayed to a user, stored for later use or further processing, or both, as indicated at step 410. For example, the predicted treatment outcome and/or suggested clinical course of action can be indicated to the adolescent patient and/or to a clinician (e.g., via a display or other user interface of a computer, a smart phone, a tablet, a wearable device, or some other system or device).

In an example study, a PGB-based model was constructed and used to predict treatment outcome in adolescent patients with MDD. In this example study, Treatment for Adolescents with Depression Study (“TADS”) and Eli Lilly Company datasets were examined. The patients in these data sets had the characteristics reported in Table 5.

TABLE 5

| Exposure and Sample | | | | |
|---|---|---|---|---|
| Characteristics | | Fluoxetine | Duloxetine | Placebo |
| Use in Analyses | | Training | Testing | Testing |
| Data Source | | TADS and | Eli Lilly | TADS and |
| | | Eli Lilly | | Eli Lilly |
| Sample Size Breakdown | | TADS: 92 | HMCK: 88 | TADS: 88 |
| | | HMCK: 95 | HMCL: 167 | HMCK: 89 |
| | | HMCL: 84 | | HMCL: 88 |
| Total Subjects | | 271 | 255 | 265 |
| Age | Range | 7 to 18 | 7 to 18 | 7 to 18 |
| (in years) | Median | 14 | 13.3 | 14 |
| | Standard | 2.9 | 3.1 | 2.6 |
| | Deviation | | | |
| Sex | Male | 134 | 131 | 122 |
| | Female | 137 | 124 | 143 |
| Race | White | 202 | 179 | 184 |
| | African American | 31 | 37 | 43 |
| | Asian | 3 | 1 | 0 |
| | American Indian/ | 13 | 31 | 17 |
| | Alaska Native | | | |
| | Hispanic | 7 | 0 | 5 |
| | Multiple | 15 | 7 | 16 |

Patients received at least 10 or 12 weeks of treatment with a study drug, fluoxetine (an SSRI), duloxetine (an SNRI), or placebo. Depressive symptoms were measured using the 17-item CDRS-R at baseline, 4 weeks, and 10 weeks (in Eli Lilly's datasets) or baseline, 6 weeks, and 12 weeks (in TADS datasets). Early dropouts or patients in the arms of the TADS study that included cognitive behavioral therapy were not included.

In this example study, PGM and prognoses rules were developed from the TADS and Eli Lilly Co (N=271) datasets (fluoxetine). The model prognostic capabilities were then tested with datasets from independent cohorts of adolescents (N=255) treated for MDD with duloxetine. Data (N=265) from patients who received a pill placebo were also examined to ascertain the prognostic effects of depression symptoms that were most likely due to drug effects.

A CDRS-R score of ≥40 was defined as active depression and a baseline inclusion criterion. Remission was defined as a CDRS-R score ≤28. Response was defined as a ≥50% reduction (or non-response as a lack of ≥50% reduction) in CDRS-R score from baseline at 4 to 6 weeks (intermediate timepoint) or 10 to 12 weeks (endpoint).

In this example study, patient stratification and symptom dynamic paths were as follows. All patients in B1 or C1 strata who achieved remission were also responders, 68% of patients in B2 or C2 strata were responders (without remission), and 94% of patients in B3 and C3 strata were non-responders. Seven symptom dynamic paths (see FIG. 2B for illustration and interpretation) that originated from each baseline stratum (Table 1) were derived in patients treated with fluoxetine (training dataset).

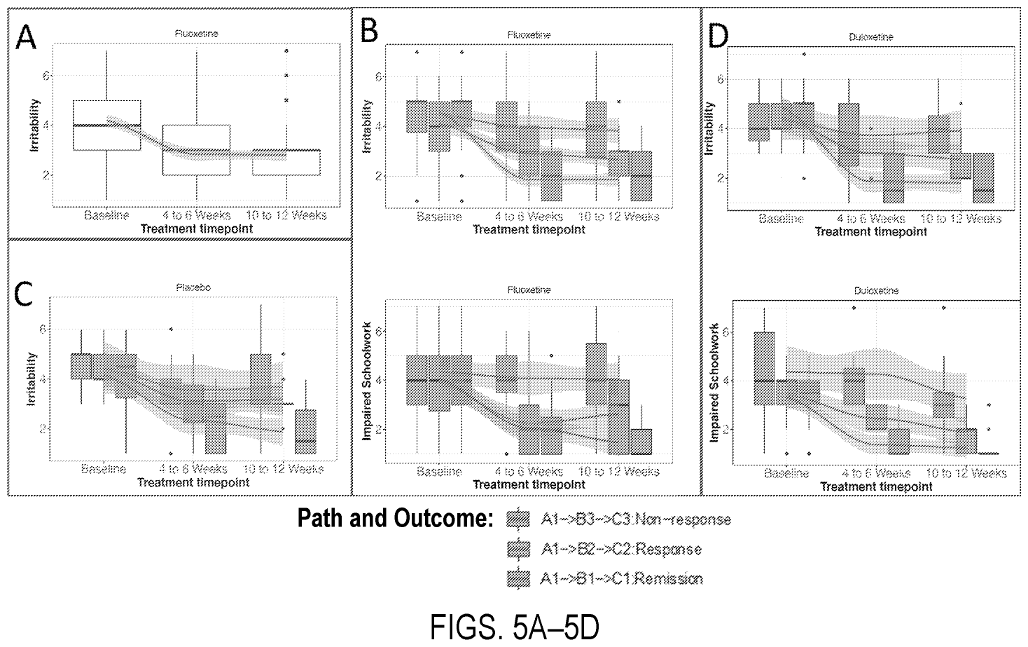

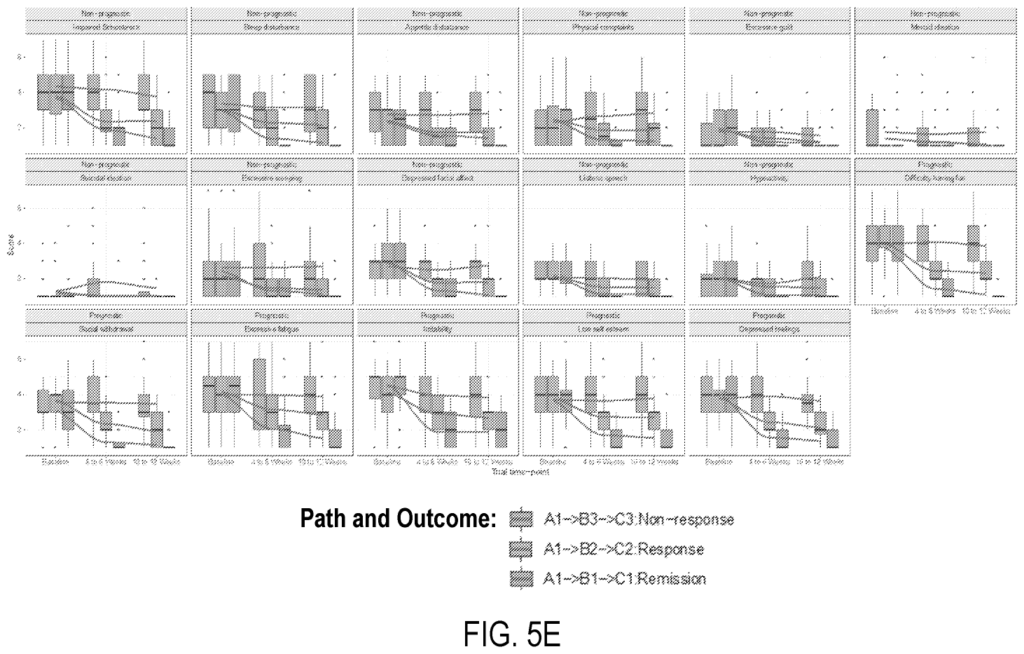

Prognostic symptoms in this example study were as follows. Six CDRS-R items (depressed feelings, difficulty having fun, irritability, low self-esteem, social withdrawal, and excessive fatigue) met the prognostic symptom criteria in the training dataset. FIGS. 5A-5D (irritability, impaired schoolwork) and FIG. 5E (all other symptoms) illustrate the variations in severity of prognostic symptoms in patients with and without the superimposition of symptom dynamic paths. In patients that are not stratified, the mean severity of irritability reduces between baseline and treatment's endpoint, but there is still a high degree of inter-patient variation in the scores (as shown by the large spread of boxplots in FIG. 5A). By stratifying patients and then deriving symptom dynamic paths (e.g., those originating from stratum A1, as shown in FIGS. 5B-5D), the discriminatory effect of scores at 10 to 12 weeks was better reflected in the patterns of response at 4 to 6 weeks. No such discriminatory effects occur for non-prognostic symptoms (e.g., impaired schoolwork) or in the prognostic symptoms (inferred from fluoxetine patients) in placebo-treated patients. No prognostic symptoms could be identified for patients who received placebo. FIGS. 5B and 5E show data for patients treated with fluoxetine, FIG. 5C shows data for patients treated with a placebo, and FIG. 5D shows data for patients treated with duloxetine.

The predictive performance of the changes in prognostic symptoms at 4 to 6 weeks from treatment initiation in this example study are summarized in Table 3.

For patients originating in the A2 stratum, the following were observed. The accuracy in the prediction of non-response at 10 to 12 weeks was 62% (OR 12.3, CI 2.5-58.3, p=0.06) by transitioning into the B3 stratum with 4 prognostic symptoms improved by 2 point at 4 to 6 weeks. Further, the accuracy in the prediction of response at 10 to 12 weeks was 86% (OR 8.4, CI 1.1-63.7, p=6.8E-05) by transitioning into the B2 stratum with 3 prognostic symptoms improved by 2 points at 4 to 6 weeks. Additionally, the accuracy in the prediction of remission at 10 to 12 weeks was 67% (OR 8, CI 0.78-81, p=0.09) by transitioning into the B1 stratum with 4 prognostic symptoms improved by 2 points at 4 to 6 weeks.

For patients originating in the A1 stratum, the following were observed. The accuracy in the prediction of non-response at 10/12 weeks was 66% (OR 7, CI 1.6-30.4, p=0.007) by transitioning into the B3 stratum with 5 prognostic symptoms improved by 1 point at 4 to 6 weeks. Further, the accuracy in the prediction of response at 10 to 12 weeks was 84% (OR 5.4, CI 0.2-7, p=0.005) by transitioning into the B2 stratum with 4 prognostic symptoms improved by 1 point at 4 weeks. Additionally, the accuracy in the prediction of remission at 10 to 12 weeks was 72% (OR 8.3, CI 0.6-7.66, p=0.05) by transitioning into the B1 stratum with 4 prognostic symptoms improved by 2 points at 4 to 6 weeks.

In over 71% of the patients starting from any of the baseline strata, the criteria for minimum number of prognostic symptoms needed for threshold rules were satisfied. The outcome was non-response for nearly all (89%) of the remaining patients.

Patients in the testing datasets who were treated with duloxetine, were assigned to a stratum at each time point, as defined by the same range of total CDRS-R scores derived from the training dataset. The same thresholds of prognostic symptom changes at 4 weeks derived from the training cohort were applied to the testing dataset to predict outcomes at outcomes at 10 weeks, as summarized in Table 4 above.

For patients originating in the A2 stratum, the following were observed. The accuracies in the prediction of non-response at 10 weeks was 63% (p=0.02) for patients treated with duloxetine who transitioned to the B3 stratum with 4 prognostic symptoms improved by 2 point at 4 weeks. Further, the accuracies in the prediction of response at 10 weeks was 84% (p=6.61E-05) for patients who transitioned to the B2 stratum with 3 prognostic symptoms improved by 2 points at 4 weeks. Additionally, the accuracies in the prediction of remission at 10 weeks was 78% (p=0.007) for patients who transitioned to the B1 stratum with 4 prognostic symptoms improved by 2 points at 4 weeks.

For patients originating in the A1 stratum, the following were observed. The accuracies in the prediction of non-response at 10 weeks was 70% (p=0.06) for patients treated with duloxetine who transitioned to the B3 stratum with 5 prognostic symptoms improved by 1 point at 4 weeks. Further, the accuracies in the prediction of response at 10 weeks was 84% (p=6.6E-05) for patients who transitioned to the B2 stratum with 4 prognostic symptoms improved by 1 points at 4 weeks. Additionally, the accuracies in the prediction of remission at 10 weeks was 70% (p=0.09) for patients who transitioned to the B1 stratum with 4 prognostic symptoms improved by 2 points at 4 weeks.

Analogous to the case in the training dataset, the prognostic symptom criteria captured variations in 77% of patients from each baseline cluster across all of the testing datasets. Nearly all (93%) of the remaining patients were non-responders at 10 weeks.

Neither age, sex, nor race were associated with chances of meeting the prognostic symptom criteria or the prediction accuracy in either fluoxetine or duloxetine treated patients.

Prognostic depressive symptoms were not identified in patients who received placebo in this example study. Table 4 summarizes the accuracy of predicting outcomes in placebo patients (assigned to baseline and 4 to 6 weeks strata) using the four prognostic CDRS-R derived symptoms and compared these outcomes to fluoxetine treatment patients (Table 3). The accuracies for predicting non-response were significantly lower in placebo patients in comparison with fluoxetine-treated patients.

This study demonstrated that PGMs are capable of generating interpretable predictions of antidepressant response in adolescents with MDD across two classes of antidepressants. Six symptom trajectories (anhedonia, social withdrawal, energy level, irritability, self-esteem, and depressed mood) after 4 to 6 weeks of treatment were predictive of clinical outcomes at 10 to 12 weeks. This preliminary work suggests that the computational models described in the present disclosure can assist clinical decisions by informing physicians on the selection, use, and dosing of antidepressants for adolescents with MDD.

In this way, the systems and methods described in the present disclosure provide a framework for a symptom-based tool to derive interpretable predictions of treatment outcomes in adolescents with depression. In addition to the patient data described above, the symptom-based PGMs can incorporate pharmacogenomic data, functional connectivity data, neural metabolomic data, genomic data, and other biological measures to enhance the predictability of treatment outcomes.

Further improvement in symptomatology-based predictability of outcomes for placebo-treated patients are advantageous for clinical and research efforts. For example, placebo responders can be treated with, structure, watchful waiting, and psychosocial treatments, which could be employed first as a treatment regimen rather than an antidepressant. Conversely, adolescents predicted to poorly respond to placebo could receive antidepressant treatment early. Accurate predictions of placebo response in adolescents with MDD could also be used to refine clinical trial methodology. For instance, protocols could specify that all placebo responders identified by PGMs exit the trial at 4 weeks. This would result in an enriched sample to examine the true effect of the active antidepressant. Early prediction and removal of placebo responders addresses a contributor to failed pharmacotherapy trials for depression in adolescents.

Predicting outcomes in adolescents treated for depression is advantageous in managing what could manifest into a lifelong disease burden. Recent studies raise many questions regarding the potential for over prescription, under prescription, and potential inequities of treatment for MDD in adolescents. To this end, efforts are underway to train and engage primary care physicians in the optimal treatment of MDD in adolescents. The algorithm-based approaches and decision support tools described in the present disclosure can enhance treatment approaches for adolescents with depression in primary care.

The use of PGMs represents an analytical improvement through the ability to derive interpretable prognoses of antidepressant treatment outcomes in depressed adolescents across broad classes of antidepressants. The patient stratification at intermediate (e.g., 4 to 6 weeks) or later (e.g., 10 to 12 weeks) timepoints demonstrates considerable ecological validity given the resulting distributions of non-responders, responders without remission, and remission.

The mathematical constructs of PGMs have several advantages over previous machine learning based approached as PGMs do not infer most-likely trajectories of disease severity by conditioning on improvements in disease severity at intermediate time-points, nor need significant domain expertise to choose and interpret paths to ensure appropriate model fitness. The mathematical framework implemented by the systems and methods described in the present disclosure can be expanded to include extended study durations and asynchronous time-points (e.g., by modeling the PGM as a Markov jump process).

The systems and methods described in the present disclosure can also be adapted for predicting treatment outcome for treatment regiments other than just antidepressant use. For example, the systems and methods could be applied for extracting response trajectories to cognitive behavioral therapy (“CBT”), or sequential CBT combined with pharmacotherapy. Several other factors that can be considered include adherence, family function, socioeconomic status, comorbidities, and family history of psychiatric illness.

PGMs coupled with unsupervised machine learning techniques provide clinically relevant predictive tools for adolescents with MDD treated with fluoxetine, duloxetine, or other treatment regiments. Additionally or alternatively, the systems and methods described in the present disclosure can be implemented to examine prospectively treated patients, account for antidepressant class, examine dosing, consider psychotherapy, and integrate potential biomarkers.

The systems and methods described in the present disclosure may be implemented using a computer, a tablet, a smart phone, or other computing device to receive, via a user interface (e.g., a touch screen, a keyboard), indications of symptom measures for an adolescent patient at respective different points in time. The computing device could then, based on the received symptom measures, generate a predicted treatment outcome or other clinical course of action and provide, via the user interface (e.g., a display), an indication of the predicted treatment outcome and/or clinical course of action.

In another example, a computing device could receive, via a user interface, an indication of one or more symptom measures for an adolescent patient. The computing device could operate to obtain additional symptom measures for the adolescent patient (e.g., a pre-treatment symptom measure) by communicating, via a communication interface, with a remote system (e.g., medical records server) to access the additional symptom measures. In yet another example, a server could receive, from a remote device (e.g., a smart phone, a tablet, a clinician's computing device) indications of one or more symptom measures for the adolescent patient at respective different points in time. The server could then, based on the received symptom measures, generate a predicted treatment outcome and/or other clinical course of action. The server could then transmit an indication of the predicted treatment outcome and/or other clinical course of action to the remote device. The remote device could then provide an indication (e.g., on a display) of the transmitted predicted treatment outcome and/or other clinical course of action.

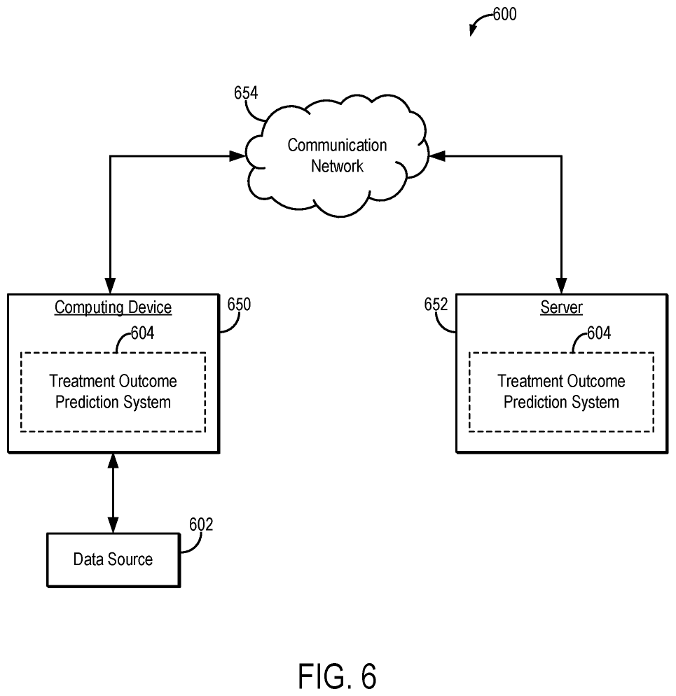

Referring now to FIG. 6, an example of a system 600 for predicting treatment outcome in adolescent patients with MDD in accordance with some embodiments of the systems and methods described in the present disclosure is shown. As shown in FIG. 6, a computing device 650 can receive one or more types of data (e.g., baseline patient data, functional connectivity data, metabolomic data, genomic data) from data source 602. In some embodiments, computing device 650 can execute at least a portion of a treatment outcome prediction system 604 to predict a likelihood of treatment outcome in an adolescent patient having MDD from data received from the data source 602.

Additionally or alternatively, in some embodiments, the computing device 650 can communicate information about data received from the data source 602 to a server 652 over a communication network 654, which can execute at least a portion of the treatment outcome prediction system 604. In such embodiments, the server 652 can return information to the computing device 650 (and/or any other suitable computing device) indicative of an output of the treatment outcome prediction system 604.

In some embodiments, computing device 650) and/or server 652 can be any suitable computing device or combination of devices, such as a desktop computer, a laptop computer, a smartphone, a tablet computer, a wearable computer, a server computer, a virtual machine being executed by a physical computing device, and so on.

In some embodiments, data source 602 can be any suitable source of data (e.g., baseline patient data, functional connectivity data, metabolomic data, genomic data), such as a database, another computing device (e.g., a server storing data), and so on. In some embodiments, data source 602 can be local to computing device 650. For example, data source 602 can be incorporated with computing device 650 (e.g., computing device 650) can be configured as part of a device for capturing, scanning, and/or storing data). As another example, data source 602 can be connected to computing device 650 by a cable, a direct wireless link, and so on. Additionally or alternatively, in some embodiments, data source 602 can be located locally and/or remotely from computing device 650, and can communicate data to computing device 650 (and/or server 652) via a communication network (e.g., communication network 654).

In some embodiments, communication network 654 can be any suitable communication network or combination of communication networks. For example, communication network 654 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), a wired network, and so on. In some embodiments, communication network 108 can be a local area network, a wide area network, a public network (e.g., the Internet), a private or semi-private network (e.g., a corporate or university intranet), any other suitable type of network, or any suitable combination of networks. Communications links shown in FIG. 6 can each be any suitable communications link or combination of communications links, such as wired links, fiber optic links, Wi-Fi links, Bluetooth links, cellular links, and so on.

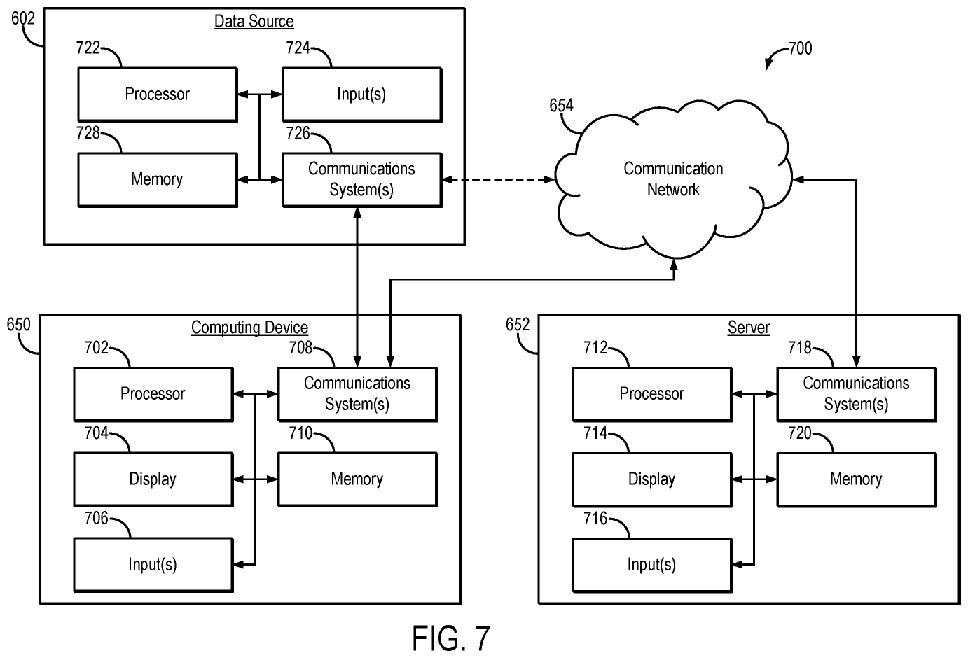

Referring now to FIG. 7, an example of hardware 700 that can be used to implement data source 602, computing device 650), and server 654 in accordance with some embodiments of the systems and methods described in the present disclosure is shown. As shown in FIG. 7, in some embodiments, computing device 650 can include a processor 702, a display 704, one or more inputs 706, one or more communication systems 708, and/or memory 710. In some embodiments, processor 702 can be any suitable hardware processor or combination of processors, such as a central processing unit (“CPU”), a graphics processing unit (“GPU”), and so on. In some embodiments, display 704 can include any suitable display devices, such as a computer monitor, a touchscreen, a television, and so on. In some embodiments, inputs 706 can include any suitable input devices and/or sensors that can be used to receive user input, such as a key board, a mouse, a touchscreen, a microphone, and so on.

In some embodiments, communications systems 708 can include any suitable hardware, firmware, and/or software for communicating information over communication network 654 and/or any other suitable communication networks. For example, communications systems 708 can include one or more transceivers, one or more communication chips and/or chip sets, and so on. In a more particular example, communications systems 708 can include hardware, firmware and/or software that can be used to establish a Wi-Fi connection, a Bluetooth connection, a cellular connection, an Ethernet connection, and so on.

In some embodiments, memory 710 can include any suitable storage device or devices that can be used to store instructions, values, data, or the like, that can be used, for example, by processor 702 to present content using display 704, to communicate with server 652 via communications system(s) 708, and so on. Memory 710 can include any suitable volatile memory, non-volatile memory, storage, or any suitable combination thereof. For example, memory 710 can include RAM, ROM, EEPROM, one or more flash drives, one or more hard disks, one or more solid state drives, one or more optical drives, and so on. In some embodiments, memory 710 can have encoded thereon, or otherwise stored therein, a computer program for controlling operation of computing device 650. In such embodiments, processor 702 can execute at least a portion of the computer program to present content (e.g., images, user interfaces, graphics, tables, treatment outcome reports), receive content from server 652, transmit information to server 652, and so on.

In some embodiments, server 652 can include a processor 712, a display 714, one or more inputs 716, one or more communications systems 718, and/or memory 720. In some embodiments, processor 712 can be any suitable hardware processor or combination of processors, such as a CPU, a GPU, and so on. In some embodiments, display 714 can include any suitable display devices, such as a computer monitor, a touchscreen, a television, and so on. In some embodiments, inputs 716 can include any suitable input devices and/or sensors that can be used to receive user input, such as a keyboard, a mouse, a touchscreen, a microphone, and so on.

In some embodiments, communications systems 718 can include any suitable hardware, firmware, and/or software for communicating information over communication network 654 and/or any other suitable communication networks. For example, communications systems 718 can include one or more transceivers, one or more communication chips and/or chip sets, and so on. In a more particular example, communications systems 718 can include hardware, firmware and/or software that can be used to establish a Wi-Fi connection, a Bluetooth connection, a cellular connection, an Ethernet connection, and so on.

In some embodiments, memory 720 can include any suitable storage device or devices that can be used to store instructions, values, data, or the like, that can be used, for example, by processor 712 to present content using display 714, to communicate with one or more computing devices 650, and so on. Memory 720 can include any suitable volatile memory, non-volatile memory, storage, or any suitable combination thereof. For example, memory 720 can include RAM, ROM, EEPROM, one or more flash drives, one or more hard disks, one or more solid state drives, one or more optical drives, and so on. In some embodiments, memory 720 can have encoded thereon a server program for controlling operation of server 652. In such embodiments, processor 712 can execute at least a portion of the server program to transmit information and/or content (e.g., data, images, a user interface, treatment outcome reports) to one or more computing devices 650, receive information and/or content from one or more computing devices 650, receive instructions from one or more devices (e.g., a personal computer, a laptop computer, a tablet computer, a smartphone), and so on.

In some embodiments, data source 602 can include a processor 722, one or more inputs 724, one or more communications systems 726, and/or memory 728. In some embodiments, processor 722 can be any suitable hardware processor or combination of processors, such as a CPU, a GPU, and so on. In some embodiments, the one or more inputs 724 are generally configured to acquire data.

Note that, although not shown, data source 602 can include any suitable inputs and/or outputs. For example, data source 602 can include input devices and/or sensors that can be used to receive user input, such as a keyboard, a mouse, a touchscreen, a microphone, a trackpad, a trackball, and so on. As another example, data source 602 can include any suitable display devices, such as a computer monitor, a touchscreen, a television, etc., one or more speakers, and so on.

In some embodiments, communications systems 726 can include any suitable hardware, firmware, and/or software for communicating information to computing device 650 (and, in some embodiments, over communication network 654 and/or any other suitable communication networks). For example, communications systems 726 can include one or more transceivers, one or more communication chips and/or chip sets, and so on. In a more particular example, communications systems 726 can include hardware, firmware and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 728 can include any suitable storage device or devices that can be used to store instructions, values, data, or the like, that can be used, for example, by processor 722 to control the one or more inputs 724, and/or receive data from the one or inputs 724; present content (e.g., images, a user interface, a treatment outcome report) using a display; communicate with one or more computing devices 650; and so on. Memory 728 can include any suitable volatile memory, non-volatile memory, storage, or any suitable combination thereof. For example, memory 728 can include RAM, ROM, EEPROM, one or more flash drives, one or more hard disks, one or more solid state drives, one or more optical drives, and so on. In some embodiments, memory 728 can have encoded thereon, or otherwise stored therein, a program for controlling operation of data source 602. In such embodiments, processor 722 can execute at least a portion of the program to generate predictive treatment outcome reports, transmit information and/or content (e.g., data, images, treatment outcome reports) to one or more computing devices 650, receive information and/or content from one or more computing devices 650, receive instructions from one or more devices (e.g., a personal computer, a laptop computer, a tablet computer, a smartphone, etc.), and so on.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (e.g., hard disks, floppy disks), optical media (e.g., compact discs, digital video discs, Blu-ray discs), semiconductor media (e.g., random access memory (“RAM”), flash memory, electrically programmable read only memory (“EPROM”), electrically erasable programmable read only memory (“EEPROM”)), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

The present disclosure has described one or more preferred embodiments, and it should be appreciated that many equivalents, alternatives, variations, and modifications, aside from those expressly stated, are possible and within the scope of the invention.

1. A computer-implemented method for predicting a treatment outcome response for an adolescent patient to a particular treatment for major depressive disorder, the method comprising:
(a) accessing symptom measure data for the adolescent patient using the computer system, wherein the symptom measure data comprise:
a first symptom measure for the adolescent patient corresponding to a severity of major depressive disorder at a first point in time;
a second symptom measure for the adolescent patient corresponding to a severity of major depressive disorder at a second point in time that is subsequent to the first point in time;
(b) selecting with the computer system, based on the symptom measure data:
a first symptom class from a first set of symptom classes corresponding to a first symptom range that contains the first symptom measure;
a second symptom class from a second set of symptom classes corresponding to a second symptom range that contains the second symptom measure;
(c) inputting the first symptom class and the second symptom class to a trained machine learning model using the computer system, generating output as a prediction of a treatment outcome for the adolescent patient in response to a particular treatment regimen; and
(d) providing an indication of the prediction of the treatment outcome using the computer system.

2. The method of claim 1, wherein the trained machine learning model comprises a probabilistic graphical model.

3. The method of claim 2, wherein the probabilistic graphical model is implemented as a hidden Markov model.

4. The method of claim 3, wherein the hidden Markov model comprises a plurality of hidden states, a plurality of observation states, and a plurality of transition probabilities.

5. The method of claim 4, wherein the hidden states comprise adolescent symptom classes defined by ranges of symptom measures.

6. The method of claim 4, wherein the observation states comprise treatment response status of adolescent patients associated with different symptom classes.

7. The method of claim 4, wherein the transition probabilities comprise fractions of adolescent patients moving between symptom classes of one timepoint to a subsequent timepoint.

8. The method of claim 4, wherein the transition probabilities can be determined using a forward algorithm to identify probable forward transitions between hidden states.

9. The method of claim 8, wherein the forward algorithm also determines the observation states as probable treatment outcomes during each transition between hidden states.

10. The method of claim 2, wherein the probabilistic graphical model comprises a plurality of nodes and at least some of the plurality of nodes are connected via symptom dynamic paths.

11. The method of claim 10, wherein the symptom dynamic paths can be determined using a forward algorithm to compute likelihoods for paths originating from a first symptom stratum and ending in a second symptom stratum without having to condition trajectories on a specific outcome of interest.

12. The method of claim 1, wherein selecting the first symptom class and the second symptom class comprise determining the first set of symptom classes and the second set of symptom classes using a Gaussian mixture model.

13. The method of claim 1, wherein the particular treatment regimen associated with the predicted treatment outcome comprises treatment with an antidepressant.

14. The method of claim 13, wherein the antidepressant is one of fluoxetine or duloxetine.

15. The method of claim 1, wherein the particular treatment regimen associated with the predicted treatment outcome comprises treatment with a placebo.

16. The method of claim 1, wherein the symptom measure data further comprise at least one of genomic data or metabolomic data obtained from the adolescent patient.

17. The method of claim 1, wherein the symptom measure data comprise Children's Depression Rating Scale-Revised (CDRS-R) total scores.

18. The method of claim 1, wherein the symptom measure data comprise Patient Health Questionnaire Modified for Teens (PHQ-9M) response data.
